(19) United States
(12) Patent Application Publication
(10) Pub. No.: US 2005/0130226 A1
(43) Pub. Date: Jun. 16, 2005

(54) Fully integrated protein lab-on-a-chip with smart microfluidics for spot array generation

(75) Inventors: Chong H. Ahn, Cincinnati, OH (US); Junhai Kai, Cincinnati, OH (US); Young-Soo Sohn, Austin, TX (US)

Correspondence Address: HAHN LOESER & PARKS, LLP, One GOJO Plaza, Suite 300, AKRON, OH, 44311-1076, US

(73) Assignee: The University of Cincinnati

(21) Appl. No.: 10/947,576

(22) Filed: Sep. 22, 2004

Related U.S. Application Data
(60) Provisional application No. 60/506,641, filed on Sep. 26, 2003.
(60) Provisional application No. 60/506,226, filed on Sep. 26, 2003.
(60) Provisional application No. 60/506,321, filed on Sep. 26, 2003.
(60) Provisional application No. 60/506,635, filed on Sep. 26, 2003.

(51) Int. Cl. H01L 21/00; G01N 33/53; G01N 33/537; G01N 33/543; C12M 1/34

(52) U.S. Cl. 435/7.1; 435/287.2; 427/2.11; 438/1

(57) ABSTRACT

Techniques for the fabrication of fully-integrated lab-on-a-chips (or biochips) specifically oriented towards point-of-care detection of biomolecules using immunoassay based detection techniques are disclosed. A primary task for the development of such biochips is the development of techniques to precisely deposit and localize the capture antibody on pre-determined locations over the biochip. The use of selective surface modification, specifically control over the surface energy, to achieve localized adsorption of the capture antibody is disclosed. Another approach, also disclosed, describes the use of smart passive microfluidics to confine the flow of the capture antibody along certain paths of the biochip and thereby control the locations over which the capture antibody is adsorbed. Furthermore, the use of an integrated microlens array as means of enhancing the detection sensitivity of the biochip is also disclosed.

### CROSS-REFERENCE TO RELATED APPLICATIONS/INCORPORATION BY REFERENCE

This application claims priority to provisional U.S. patent applications Ser. Nos. 60/506,641; 60/506,226; 60/506,321; 60/506,424; and 60/506,635 all filed on Sep. 26, 2003, and all of which are incorporated herein by reference in their entirety.

This patent application is being filed concurrently with U.S. Patent Applications having attorney docket numbers 200057.00008, 200057.00012, 200057.00010, and 200057.00011, which are incorporated herein by reference in their entirety.

### TECHNICAL FIELD

Embodiments of the present invention generally relate to the development of protein lab-on-a-chips for point-of-care monitoring applications based on immunoassay techniques. More specifically, techniques for precise patterning of immunoassay components, namely, the capture antibody are disclosed. Also disclosed is a fully-integrated protein chip, which integrates the microfluidics and biosensing components on a low-cost, plastic substrate to realize disposable protein chips. The protein chips contains a smart passive microfluidic system, a bio-functionalized binding surface as sensing site, and an optical lens array for collecting a fluorescence signal from an immunoassay test.

### BACKGROUND OF THE INVENTION

The use of microfluidic devices based on MEMS (Micro Electro Mechanical Systems) has significantly enhanced the performance of many biosensing applications. Microfluidic systems typically require ultra-small sample volumes, offer rapid reaction time and are cheaper to operate compared to their macro counterparts. Furthermore, microfluidic devices can be designed in a modular fashion and subsequently integrated to achieve a higher degree of functionality.

Protein chips have been developed as part of the development of the microfluidic MEMS (also referred to as BioMEMS devices). Protein chips have been developed for detecting a wide variety of proteins. One such set of proteins (also referred to as “protein biomarkers”) are the cardiac protein biomarkers. Cardio vascular diseases account for a significant number of mortalities in the United States especially in the 50-65 age range. A rapid and early detection of cardiac disorders can substantially alleviate the risk by proper medical care prior to the onset of cardiac arrest. Cardiac disorders are usually accompanied by the release of specific peptides and/or proteins into the blood stream that can serve as indicators of a serious impending cardiac event. A number of cardiac proteins have been identified including Troponin, C-reactive protein, Myoglobin, Fatty Acid Binding Protein (FABP), Glycogen Phosphorylase Isoenzyme BB (GPBB), NT-proBNP/proBNP etc.

Most protein lab-on-a-chips or biochips use some form of immunoassay based techniques. Immunoassay based detection techniques make use of the unique recognition between antibodies and antigens (antigens being the target molecule of interest). For such techniques, the most common approach is described briefly as follows: a primary antibody is first coated on a suitable substrate; then the antigen is introduced and selectively binds to the primary antibody; then a secondary antibody is introduced which binds selectively to another site on the antigen. The secondary antibody is also “labeled” with either a flurophore or an enzyme. The flurophore can be directly detected by fluorescent detection techniques, whereas the enzyme is used to catalyze a substrate and the outcome of the reaction can be used to monitor the concentration of the antigen. This approach is called the “sandwich” immunoassay and most commonly used. In some cases, single step or “competitive” immunoassays are used, though herein, the term immunoassay refers to the sandwich immunoassay protocol. Furthermore, to date most devices referred to as “protein biochip” typically incorporate high-density arrays of primary antibodies deposited on a substrate in close proximity. The subsequent steps of the assay are carried out on the open substrate surface (although in some cases micro-structured depressions may be used) with the assistance of robotic dispensing systems. The critical tasks in this application include: (a) suitable technique for binding the primary antibody to the substrate and (b) confining the binding areas of the primary antibodies. It is worth noting that this approach is not well suited for point-of-care testing apparatus because of the bulky nature of the fluid handling equipment.

There have been a number of different approaches to effectively address the issue of binding the primary antibody on to the substrate. The simplest approach is direct deposition of the protein on the substrate and coupling via non-specific adsorption. U.S. Pat. No. 6,475,809 and U.S. Pat. No. 5,620,850 (incorporated herein in their entirety by reference) discuss the use of SAM's (self assembled monolayer) for the use of primary antibody adsorption on the substrate. U.S. Pat. No. 5,925,552 (incorporated herein in its entirety by reference) discusses the use of specialized cross-functional groups that react with the substrate and the desired capture antibody and can act as effective coupling agents whereas U.S. Pat. No. 5,242,828 (incorporated herein in its entirety by reference) discusses the use of specialized ligand groups for this application. Such approaches are well known in the art.

A more difficult issue to address has been the precise patterning of the capture antibody wherein, the capture antibody can be precisely confined to certain sections of the substrate and used subsequently for immunoassay based detection of the target antigen. As mentioned previously, the most prevalent application of such protein chips is for HTS (high-throughput screening) applications wherein a large number of unknowns are to be tested simultaneously. As such, it is desirable to localize the capture antibody to as small an area as possible and furthermore to arrange an array of similar or dissimilar capture antibodies in close proximity to each another on the same substrate. Perhaps the most common approach to achieve this is via the use of robotic dispensing systems, in which the substrate is physically transposed in minute increments and minute volumes of the capture antibody are deposited over the substrate as described in G. Kovacs, “Micromachined Transducers Sourcebook”, WCB-McGraw Hill, New York, 1998. Other innovative approaches include the use of specialized polymeric fibrous supports (which are located only in certain areas of the substrate) as disclosed in U.S. Pat. No. 4,855,234 (incorporated herein in its entirety by reference). U.S. Pat. No. 6,730,516 and U.S. 20030111599A1 (incorporated herein in their entirety by reference) describe the use of microfabricated nozzle arrays to deposit the capture antibody in specified locations. In all the references listed above, none of the techniques have been designed for a lab-on-a-chip specifically oriented towards point-of-care application. Most if not all the work in protein biochips has primarily been focused towards the development of HTS proteomics screening tools.

In terms of the detection techniques, the most favored approach has been the use of Enzyme based detection techniques such as ELISA (enzyme linked immunosorbent assay) for conventional bench-top immunoassay based analysis equipment. For micro-scale analysis tools, fluorescent labels and subsequent fluorescence detection of the immunoassay as described in G. Kovacs, “Micromachined Transducers Sourcebook”, WCB-McGraw Hill, New York, 1998 are most commonly used. Chemiluminescence techniques are widely accepted to be more sensitive due to the inherent self-amplification capability of the enzymes (attached to the secondary antibody) as described in Julia Yakovleva et al, Biosensors and Bioelectronics 19 (2003) 21-34 and U.S. 20020123059A1 (incorporated herein in its entirety by reference). In a few cases, alternate detection techniques such as electrochemical detection have also been employed as described in Joel S. Rossier et al, Lab on a Chip, 2001, 1, 153-157.

With the rapid developments in microfluidics technology, recently there is also considerable interest in the development of integrated lab-on-a-chip for diagnostic applications as evidenced by the study of immunoassay performance in microchannels described in Joel S. Rossier et al, Langmuir 2000, 16, 8489-8494. One of the earliest demonstrations of an integrated meso-scale system is described in U.S. Pat. No. 5,866,345 wherein all the fluidic control and also detection components are built into single functional analysis equipment. U.S. 20020123059A1 describes an integrated biochip which incorporates on-chip reagent storage, fluidic control and detection on a microfabricated platform. However, the microfabricated biochip is still used in conjunction with large analysis equipment that limits its mobility. An innovative approach is described in WO04062804A1 wherein the reagents are stored in a series of breakable pouches and furthermore incorporates fluidic control by sequentially rupturing the pouches and releasing the fluids. None of the approaches described above, however present a truly integrated biochip that addresses all the issues in the development of disposable lab-on-a-chip for diagnostic screening of proteins.

Based on the above discussion it is obvious that there is clear need to develop a more comprehensive set of techniques that can be used for the development of a low-cost, disposable biochip for point-of-care testing applications.

### SUMMARY OF THE INVENTION

Recently, a novel fabrication process that is suitable for such an approach has been demonstrated by J. Kai et al, “Study on Protein (IgG) Adsorption in Terms of Surface Modification of Cyclic Olefin Copolymer (COC) for Protein Biochip,” Proceedings of the 6th International Conference on Micro Total Analysis Systems (micro-TAS 2002), Nara, Japan, Nov. 3-7, 2002, pp. 419421, also J. Kai et al, “Protein Microarray on Cyclic Olefin Copolymer (COC) for Disposable Protein Lab-on-a-Chip,” Proceedings of the 7th International Conference on Micro Total Analysis Systems (micro-TAS 2003), California, USA, Oct. 5-9, 2003, p. 1101-1104, and J. Kai et al, “An Ultra-Fast Immunoassay in Protein Lab-on-a-Chips on Cyclic Olefin Copolymer (COC)”, Proceedings of the 8th International Conference on Micro Total Analysis Systems (micro-TAS 2004).

A protein chip is disclosed that, in accordance with an embodiment of the present invention, is designed to sense four of the cardiac markers namely; C-reactive protein, Myoglobin, Cardiac troponin (Tn L and Tn T), BNP (B-Natriuretic Peptide/ProBNP) although in principle the disclosed protein chip may be extended to the detection of other cardiac markers and proteins also. The sensing principle relies on selective binding of antibodies at specific sites on the biochip achieved by surface modification of the COC (cyclic olefin copolymer) substrate. In one embodiment of the invention, a protein microarray is created by selective surface modification of a plastic substrate followed by exposure of the substrate to antibodies. The antibodies selectively bind to specific areas of the microarray following which, the target proteins are bound to the antibodies finally followed by tagging with a fluorescent molecule. The use of selective surface modifications avoids the use of high cost and complex robotic dispensing systems and allows for developing low cost protein chips.

Also disclosed herein are techniques for the patterning of the capture antibody using a bio-functionalized surface and the subsequent use of this for the development of a fully-integrated disposable biochip for point-of-care testing applications.

A first technique involves selective surface modification using Plasma RIE (reactive ion etching) techniques. In this case, selected areas of the substrate, which is a plastic in accordance with an embodiment of the present invention, are treated to be strongly hydrophilic. It is observed that the antibodies are not adsorbed on strongly hydrophilic surfaces hence this method may be used for determining the precise location of antibody adsorption. In accordance with an embodiment of the present invention, a surface of a plastic substrate is selectively modified to form a plurality of hydrophobic spots on a hydrophilic background. The plurality of spots are formed by performing photolithography to distinguish the spots from the background on the surface of the substrate and then performing reactive ion etching to change a contact angle of the surface such that the spots are hydrophobic and the background is hydrophilic.

A second technique involves the use of smart microfluidic design, wherein capillary forces are used to preferentially direct flow in certain areas of the biochip, wherein at least some portion of the flow path overlaps the detection region. As the primary antibody solution passes through the smart microfluidic channels, the primary antibodies are adsorbed on the sidewalls and in the region where they overlap the detection region. Subsequent immunoassay steps may be used to detect relevant antigens.

A third technique involves the use of an array of planar microdispensers to deliver a precise volume of the primary antibody solution to an exact location on the biochip. Furthermore, this design can be modified wherein the microdispenser arrays dispense the liquid volume on to another substrate using a 3-D configuration. It is envisaged that the latter arrangement is more conducive for subsequent immunoassay sequencing.

Also disclosed herein is the concept of a fully-integrated biochip wherein all the relevant biochip functions namely: on-chip storage of reagents, microfluidic movement, microfluidic sequencing, immunoassay steps, and detection are incorporated on to the same biochip to realize a truly stand-alone biochip which can be immediately applied for point-of-care testing.

Furthermore, also disclosed herein is the use of an integrated microlens array which is used to enhance the fluorescent or chemiluminescence signal obtained from the immunoassay reactions.

Without intent of limiting the scope of application of the present invention, certain embodiments of the present invention are generally a low-cost, disposable plastic biochip for the analysis of cardiac biomarkers that serve as early indicators of cardiovascular disease. It will be apparent from the disclosure that the present invention is not limited to this application and indeed may be applied for the detection of virtually any peptide/protein or other biomolecule for which immunoassay detection techniques may be employed.

Certain embodiments of the present invention overcome the deficiencies and inadequacies in the prior art as described in the previous section and as generally known in the industry.

Certain embodiments of the present invention provide a means for controlling the area over which the capture antibody is deposited by appropriate control of the surface energy of certain regions of the biochip. Furthermore it is the intent herein that the technique may be used for the development of fully-integrated disposable biochips for protein analysis.

Certain embodiments of the present invention allow for the development of a smart, passive microfluidic configuration wherein the capture antibody may be deposited in selected areas of a pre-determined detection region. Furthermore, this technique may also be used as a part of a fully integrated, disposable biochip for protein analysis.

Certain embodiments of the present invention integrate smart microfluidic dispenser arrays as a part of the biochip and thereafter these dispenser arrays to be used for controlling the regions over which the capture antibody is localized and adsorbed.

Certain embodiments of the present invention allow for the development of a fully integrated protein biochip which may be operated with minimal external controls and is suitable for use with a miniature (handheld or smaller) analyzer for the analysis of an array of proteins, peptides, DNA sequences, DNA adducts, cells, viral cells, or other biomolecules than may be detected using immunoassay techniques.

Certain embodiments of the present invention provide a means for signal enhancement, thereby improving the sensitivity of detection, for the biochip by integrating a microlens array with the biochip.

Other embodiments, features, and advantages of the present invention will become apparent from the detailed description of the invention when considered in conjunction with the accompanying drawings.

### BRIEF DESCRIPTION OF THE DRAWINGS

The present invention, as defined in the claims, can be better understood with reference to the following drawings. The drawings are not all necessarily drawn to scale, emphasis instead being placed upon clearly illustrating principles of the present invention.

FIGS. 1a-1b show a schematic sketch of an embodiment of a fully-integrated disposable biochip on a plastic substrate incorporating all the functionality required for point-of-care testing of various biomolecules using immunoassay techniques, in accordance with various aspects of the present invention.

FIGS. 2a-2f show a technique for controlling the area and location of the capture antibodies using a plasma RIE process, in accordance with an embodiment of the present invention.

FIGS. 3a-3g show various configurations of a smart passive microfluidic arrangement for selectively depositing the capture antibody in pre-defined areas of the detection chamber, in accordance with various embodiments of the present invention.

FIGS. 4a-4c show the use of integrated microdispenser arrays for controlling the volume, location and delivery sequence of the capture antibodies, in accordance with an embodiment of the present invention.

FIG. 5 shows an integrated microlens array for enhancing the signal from a biochip thereby increasing the sensitivity of detection, in accordance with an embodiment of the present invention.

### DETAILED DESCRIPTION OF THE INVENTION

Described herein are techniques to fabricate and develop fully-integrated disposable biochips specifically oriented towards the detection of biomolecules using immunoassay detection techniques. In accordance with an embodiment of the present invention, a biochip is used for detecting an array of cardiac biomarkers (proteins and peptides). Furthermore, disclosed herein are techniques to increase the sensitivity of the biochip by the use of an integrated microlens array.

Definitions

The process of “Microfabrication” as described herein relates to the process used for manufacture of micrometer sized features on a variety of substrates using standard microfabrication techniques as understood widely by those skilled in this art. The process of microfabrication typically involves a combination of processes such as photolithography, wet etching, dry etching, electroplating, laser ablation, chemical deposition, plasma deposition, surface modification, injection molding, hot embossing, thermoplastic fusion bonding, low temperature bonding using adhesives and other processes commonly used for manufacture of MEMS (microelectromechanical systems) or semiconductor devices. “Microfabricated” or “microfabricated devices” as referred to herein refers to the patterns or devices manufactured using the microfabrication technology.

The term “chip”, “microchip”, or “microfluidic chip” as used herein means a microfluidic device generally containing a multitude of microchannels and chambers that may or may not be interconnected with each another. Typically, such biochips include a multitude of active or passive components such as microchannels, microvalves, micropumps, biosensors, ports, flow conduits, filters, fluidic interconnections, electrical interconnects, microelectrodes, and related control systems. More specifically, the term “biochip” is used to define a chip that is used for detection of biochemically relevant parameters from a liquid or gaseous sample. The microfluidic system of the biochip regulates the motion of the liquids or gases on the biochip and generally provides flow control with the aim of interaction with the analytical components, such as biosensors, for analysis of the required parameter. As used herein, the terms “biochip” and “lab-on-a-chip” are used interchangeably.

The term “microchannel” as used herein refers to a groove or plurality of grooves created on a suitable substrate with at least one of the dimensions of the groove being in the micrometer range. Microchannels can have widths, lengths, and/or depths ranging from 1 μm to 1000 μm. It should be noted that the terms “channel” and “microchannel” are used interchangeably in this description. Microchannels can be used as stand-alone units or in conjunction with other microchannels to form a network of channels with a plurality of flow paths and intersections.

The term “microfluidic” generally refers to the use of microchannels for transport of liquids or gases. A microfluidic system includes a multitude of microchannels forming a network and associated flow control components such as pumps, valves and filters. Microfluidic systems are ideally suited for controlling minute volumes of liquids or gases. Typically, microfluidic systems can be designed to handle fluid volumes ranging from the picoliter to the milliliter range. The term “smart microfluidics” implies a microfluidic channel network wherein a certain sequence of microfluidic operations is programmed through the use of the “Structurally Programmable Microfluidic System or sPROMs” approach in conjunction with solid-propellant based fluidic actuators. The sPROMs technology is described in detail in U.S. Provisional Patent Applications Ser. No. 60/204,214 filed on May 12, 2000 and 60/209,051 filed on Jun. 2, 2000, both of which are incorporated herein by reference in their entirety.

The term “substrate” as used herein refers to the structural component used for fabrication of the micrometer sized features using microfabrication techniques. A wide variety of substrate materials are commonly used for microfabrication including, but not limited to silicon, glass, polymers, plastics, and ceramics to name a few. The substrate material may be transparent or opaque, dimensionally rigid, semi-rigid or flexible, as per the application they are used for. Generally, microfluidic devices consist of at least two substrate layers where one of the faces of one substrate layer contains the microchannels and one face of the second substrate layer is used to seal the microchannels. The terms “substrate” and “layer” are used interchangeably in this description. Specifically, the substrate is a material that can withstand the thermal dissociation temperature of solid-propellant materials.

The term “UV-LIGA” describes a photolithography process modeled on the “LIGA” fabrication approach. LIGA refers to the microfabrication process for creating microstructures with high aspect ratio using synchrotron radiation and thick photoresists (ranging in film thickness from 1 μm to 5 mm). The LIGA process is used to form a template that may be used directly or further processed using techniques such as electroplating to create the microfluidic template. UV-LIGA uses modified photoresists that may be spin coated in thicknesses of 1 μm to 1 mm and are sensitive to UV radiation. UV radiation sources are commonly used in microfabrication facilities and hence UV-LIGA offers a lower cost alternative to LIGA for fabrication of high aspect ratio microstructures.

The term “master mold” as used herein refers to a replication template, typically manufactured on a metallic or Silicon substrate. The features of the master mold are fabricated using the UV-LIGA and other microfabrication processes. The microstructures created on the master mold may be of the same material as the master mold substrate e.g. Nickel microstructures on a Nickel substrate or may be a dissimilar material e.g. photoresist on a Silicon surface. The master mold is typically used for creating microfluidic patterns on a polymer substrate using techniques such as hot embossing, injection molding, and casting.

The term “bonding” as used herein refers to the process of joining at least two substrates, at least one of which has microfabricated structures, e.g. a microchannel, on its surface to form a robust bond between the two substrates such that any liquid introduced in the microchannel is confined within the channel structure. A variety of techniques may be used to bond the two substrates including thermoplastic fusion bonding, liquid adhesive assisted bonding, use of interfacial tape layers, etc. Specifically in this description the terms “bonding” and “thermoplastic fusion bonding” are used interchangeably. Thermoplastic fusion bonding involves heating the two substrates to be joined to their glass transition temperature and applying pressure on the two substrates to force them into intimate contact and cause bond formation. Another bonding process, namely the use of UV-adhesive assisted low temperature bonding, is also described herein and is specifically and completely referred to in all occurrences.

The term “solid-propellant” as used herein refers to any material that can liberate a substantial volume of gas upon direct heating. The liberated gas may be biochemically reactive such as Oxygen or a biochemically inert gas such as Nitrogen. A wide variety of solid-propellants are available commonly with varying properties in terms of physical structure, i.e. liquid or solid, chemical composition, dissociation temperature, chemical structure of released gas, volume of released gas and so on. The choice of a suitable propellant is governed by a number of factors such as chemical nature of the evolved gas, volume of evolved gas, dissociation temperature, and toxicity or lack thereof of the gaseous and non-gaseous components after dissociation. In this description, azobis-isobutyronitrile (AIBN) is described as a used solid-propellant, however it is understood that any suitable solid-propellant that matches the characteristics stated above for the given application may be substituted instead of AIBN and the scope of the present invention is not limited to this particular material.

The term “micropump” as used herein, refers to a device or arrangement that can provide force for displacement of liquids or gases entrapped within a microchannel. A wide variety of pumping mechanisms are known in the art and specifically in this description the “micropump” is of a positive displacement type wherein the pump generates a positive pressure, above the atmospheric pressure, and the higher pressure is coupled to one of a microfluidic column via suitable fluidic interconnects and microchannels. The differential pressure causes movement of the liquid plug or column. An “integrated micropump” or “integrated pressure source” or “on-chip micropump” or “on-chip pressure source” as used herein, refers to a micropump configuration that is irreversibly attached to or is an integral part of the microfluidic chip. The above listed terms are used interchangeably in this description.

The “functional on-chip pressure generator” or “functional pressure generator” or “functional on-chip pressure generator using solid-propellant” as used herein, are used interchangeably, and refer to a positive pressure source whose output, i.e. the pressure, can be dynamically regulated after the pressure source has been fabricated and assembled or integrated with the biochip.

The term “immunoassay” as used herein refers to a biochemical detection process that relies on the selective binding ability between various antibodies and their target antigens. Immunoassays as described herein refer to the sandwich immunoassay protocol, which is well known in the art.

The term “primary antibody” as used herein, refers to a component of the sandwich immunoassay. Typically, the “primary” or “capture” antibody is positioned at a pre-determined location on a substrate and subsequently exposed to an array of antigens. Only the antigens associated with the capture antibody will combine irreversibly with the antibody. The terms “primary antibody” and “capture antibody” are used interchangeably in this description.

The term “secondary antibody” refers to the signaling component of the immunoassay sandwich. The secondary antibody is labeled with a fluorescent dye (in the case of fluorescent detection) or with an enzyme (for electrochemical or ELISA or chemiluminescent detection). The secondary antibody will selectively bind with the antigens (which are typically already bound to the primary antibody and thus fixed to the substrate), and is then subsequently interrogated using an appropriate technique.

The term “fluorescent detection” refers to a process wherein, excitation is supplied in the form of optical energy to a particular molecule which will then absorb the energy and subsequently release the energy at another wavelength. The fluorescent detection technique requires the use of an excitation source, excitation filter, detection filter and detector. The term “chemiluminescence” refers to a process wherein certain molecules when catalyzed in the presence of an enzyme, undergo a specific biochemical reaction and emit light at a particular wavelength as a result of this reaction. Chemiluminescent detection techniques only require a detector without the need for an excitation source or filters.

The term “biomarkers” as used in this description refer to a biomolecule that is generated in response to a specific physiological condition. For example, muscular stress injuries cause the release of a biomarker called CRP whereas cardiovascular injuries cause the liberation of Cardiac Troponins. Biomarkers may or may not be uniquely associated with a particular physiological condition.

The intent of defining the terms stated above, is to clarify their use in this description and does not explicitly or implicitly limit the application of the present invention by modifications or variations in perception of the definitions.

Fully Integrated Protein Lab-On-A-Chip with Smart Microfluidics

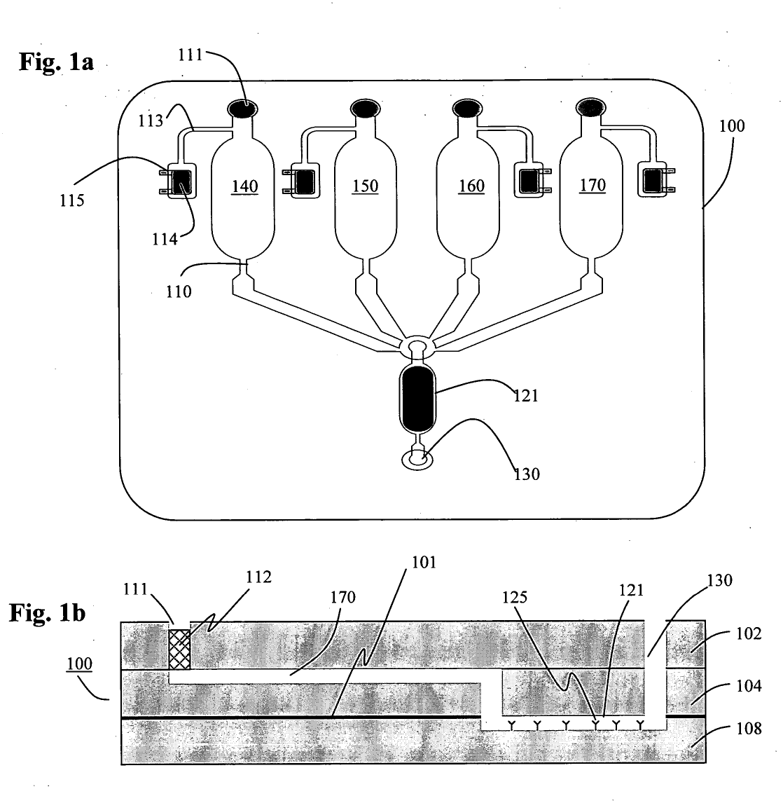

FIGS. 1a-1b show a schematic illustration of a fully integrated, disposable protein lab-on-a-chip on a plastic substrate, in accordance with an embodiment of the present invention. COC (Cyclic Olefin Copolymer) is chosen as the substrate material owing to its excellent biocompatibility properties, in accordance with an embodiment of the present invention.

As shown in FIG. 1a and FIG. 1b, the lab-on-a-chip or biochip 100 comprises multiple layers. The top layer 102 is used only for sealing for the microfluidic structures on layer 2 104. The top layer 102 also contains self-sealing inlets 111 and outlets 130 for microfluidic flow. The self sealing inlets 111 are composed of a hole plugged by a soft, deformable material 112 (such as Silicone). When a needle is used to load the sample in the biochip, the soft plug material allows easy penetration and, due to its elastic properties, will seal up the hole and prevent backflow when the needle is withdrawn.

The second layer houses most of the microfluidic and microfluidic flow control structures. The four reservoirs 140, 150, 160, and 170 are used to contain buffer solution, secondary antibody solution, sample, and an enzyme substrate solution (in the case of chemiluminescence detection). Each reservoir is also connected to a solid-propellant chamber via a narrow channel 113. The narrow channel inhibits flow into the solid-propellant chamber during filling. Furthermore, each reservoir has a passive valve 110 at its distal end which ensures that, at the correct fill pressures, the liquid does not overflow out of the reservoirs.

The third and bottom layer 108, houses the reaction chamber 121. The bottom surface of the reaction chamber is coated with the capture antibody 125 using a variety of techniques described later in the proposal. If the primary antibody is already coated on areas of the bottom layer 108 prior to assembly, a room-temperature UV-assisted (or some other form of low temperature adhesive) 101 is used to assemble the bottom layer to the top two layers. The room-temperature bonding ensures that the antibodies are not damaged due to heat.

During biochip fabrication, initially the biochip is assembled (with or without the capture antibodies in the detection area). Then, the buffer, secondary antibody and enzyme substrate solution are loaded into their respective reservoirs and the biochip is sealed. In accordance with an embodiment of the present invention, two layers of metal tape are used to completely ensconce the biochip. The entire biochip is then frozen, for example, at −20° C. or at any temperature lower than 0° C. The freezing process ensures two goals: the biologically active compounds (namely the antibodies and enzymes) are stable during storage and that there is no fluid loss due to evaporation.

For operation, the biochip is first “thawed” and all frozen components are reconstituted to a liquid state. Then the sample solution is injected into the appropriate reservoir. Note that the sampling reservoir may also be coupled to a microneedle for direct sampling as described in U.S. Patent Application with attorney docket number 200057.00010. Then, the biochip is inserted into a suitable analyzer which provides electrical contacts to the solid-propellant electrodes and also houses the optical detection system. After this, the solid-propellant material 114 in the chamber adjacent to the sample reservoir is actuated by a applying a current pulse to the heater 115. As described in U.S. Patent Application with attorney docket number 200057.00011, this causes a release of gas which builds up pressure and pushes the sample to the detection chamber. The solid-propellant actuation technique is capable of precise displacement and is of great advantage for microfluidic devices since it consumes very little power.

The target antigens in the sample couple with the capture antibody during the incubation period. Owing to the high-surface area to volume ratio of microfluidic devices, the incubation times are considerably short. In our experiments, we have determined that for microchannels (or detection reservoirs) with a height of approximately 100 μm, an incubation time of approximately 1 minute leads to binding of almost all of the antigens in solution to the capture antibodies on the detection reservoir surface. Accordingly, in accordance with an embodiment of the present invention, the sample is incubated for approximately 1 minute, though shorter or longer incubation times may be used depending upon the exact configuration of the microfluidic structures.

After sample incubation, the buffer solution is introduced into the detection reservoir by activating the suitable solid-propellant as described previously. This flushes out the unbound antigens and the remaining sample. Then, the labeled secondary antibody solution (labeled, in accordance with an embodiment of the present invention, with an enzyme suited to chemiluminescence detection) is conjugated with the (antigen: capture antibody) complex. Again, buffer is used to flush out excess secondary antibodies (note that the buffer reservoir is considerably larger than the other reservoirs in the actual device). Then the substrate material is added and is catalyzed by the enzyme to produce emissions at a distinct optical wavelength. These emissions are detected by a suitable photo detector and are used to quantify the concentration of the target antigen.

As is readily apparent from the preceding discussion, the control system required for biochip operation comprises only a timer circuit coupled to power transistors or FET's which provide a short current pulse at preset intervals to fire the solid-propellant actuators thereby causing precise fluidic displacement. The detection circuit comprises a suitable photo detector, an example of which being a super-sensitive photo CCD (charge coupled device) which in turn is hooked to suitable solid-state amplifiers. This arrangement may be easily accommodated into a handheld (or even smaller) analyzer and is uniquely suited towards point-of-care testing applications. Furthermore, by fabricating this biochip out of a relatively low cost polymer substrate using mass-production techniques such as injection molding, it is possible to develop such biochips at very low cost thereby allowing their use as disposable components of a point-of-care testing system.

A notable contribution herein is the precise “patterning” of the capture antibodies on predetermined areas of the plastic substrate thereby allowing for the antibodies to be deposited only in areas of the detection reservoir.

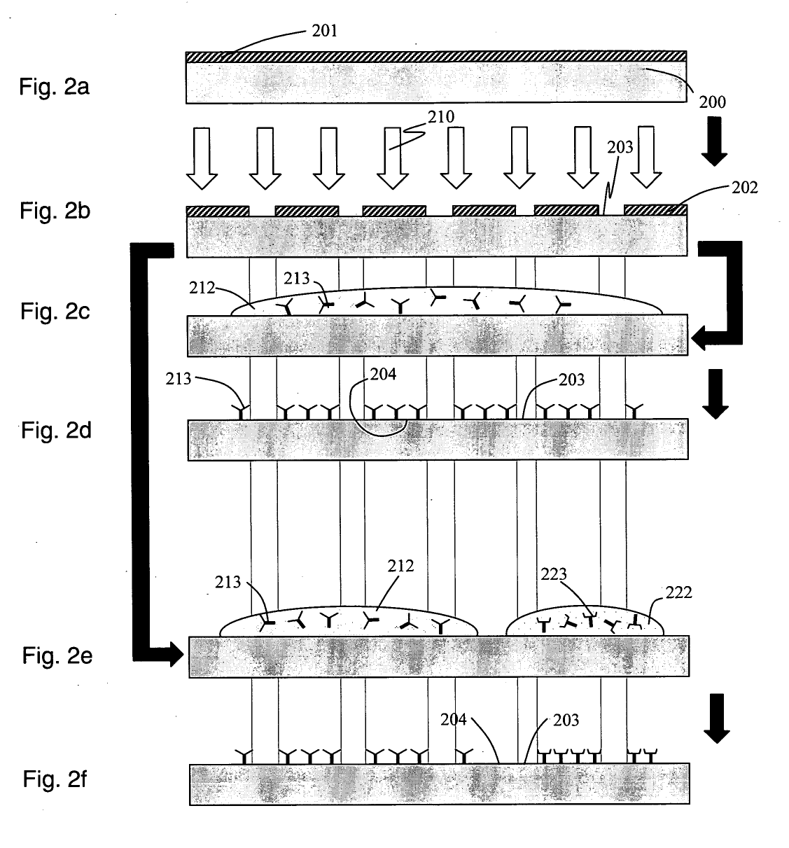

One technique for protein patterning is shown in FIGS. 2a-2f. As shown in FIG. 2a, a suitable plastic substrate 200 is initially coated with a photoresist material 201. The photoresist film 201 is patterned using well known photolithography techniques to create opening 203 in the photoresist film and leave some areas of the substrate covered with photoresist 202. As mentioned previously, in accordance with an embodiment of the present invention, the substrate material used is COC. COC has a native contact angle of ˜92° and shows very high protein adsorption on its native surface. The adsorption characteristics of COC are strongly dependent on its surface contact angle and almost no adsorption is observed on hydrophilic COC surfaces with very low contact angles (or at high surface energies).

After patterning the photoresist, the entire substrate is exposed to high frequency oxygen plasma. In accordance with an embodiment of the present invention, 13.5 MHz oxygen plasma is used for surface modification. However, it is well known in the art that other gases, including most noble gases, can also be used to render a surface hydrophilic. The exact composition of the plasma is not a novel feature and any suitable plasma treatment that can render the surface strongly hydrophilic may be used. Indeed, even alternate techniques such as selective surface coatings may be used to achieve the same goals without departing from the novelty of the present invention. Regardless of the choice of method, the sections of the substrate not covered by photoresist are rendered hydrophilic whereas other areas are maintained at the native condition (e.g., hydrophobic).

Then the capture antibody solution 212 with the desired capture antibodies 213 is used to cover the entire substrate. Again, for this, a wide variety of techniques may be used such as direct deposition from a dropper or dispenser, dip coating etc. towards the same result. The capture antibodies 213 will only adsorb to the non-plasma treated (e.g., hydrophobic) areas 204 of the substrate. As shown in FIG. 2d, this technique may also be used to coat multiple antibodies in physically different locations in the biochip by depositing different antibody solutions 213, 223 containing different antibodies 212, 222 thereby realizing a substrate with more than one type of capture antibody.

Since this approach relies on lithography techniques for defining the antibody adsorption zones, it is possible to define these locations with great accuracy and furthermore create a super-high density array of adsorbed/non-adsorbed areas. Indeed, most MEMS fabrication facilities can easily pattern these areas with dimension: spacing of 5 μm:5 μm respectively. By choosing the appropriate lithography tools, e.g. nano e-beam lithography, it is also possible to generate patterns down to a few nanometer dimensions. Also, the substrate created using this approach may be bonded to the biochip either before or after primary antibody deposition. If the primary antibody is already deposited, then a low-temperature bonding should be used. Alternatively, the substrate can be first assembled into the biochip and then the primary antibody solution may be injected in to the detection area to achieve the same results.

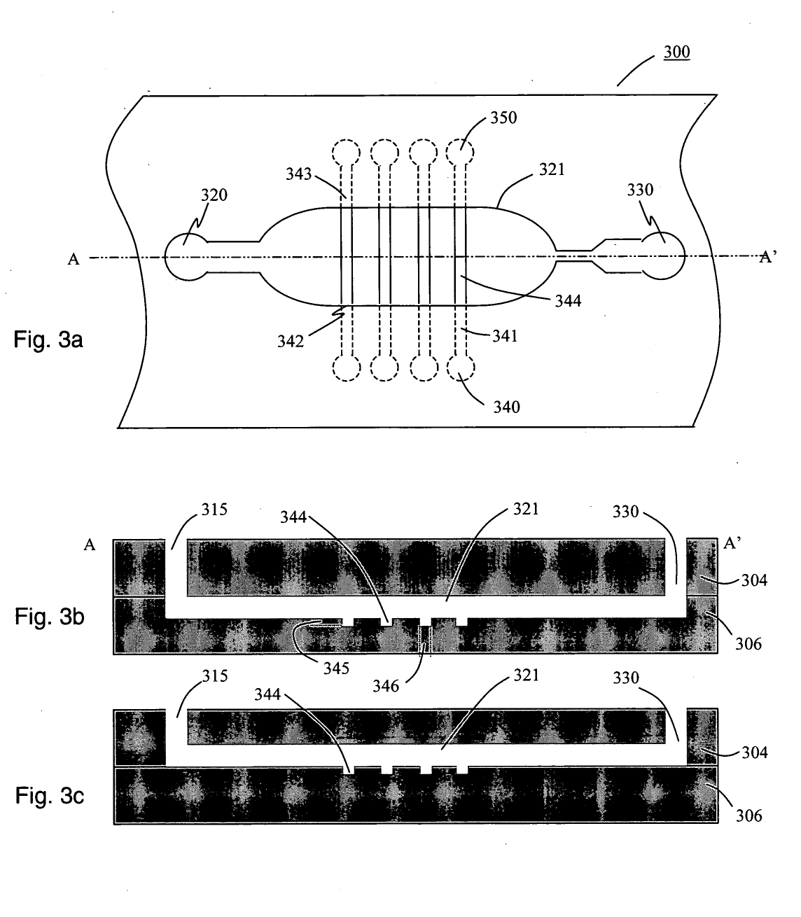

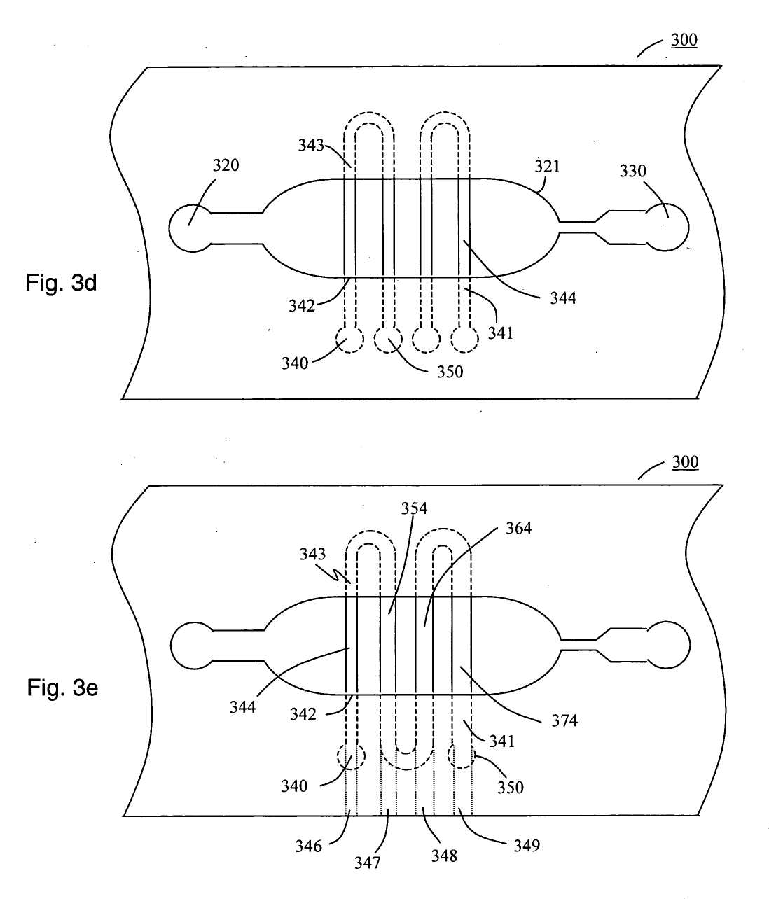

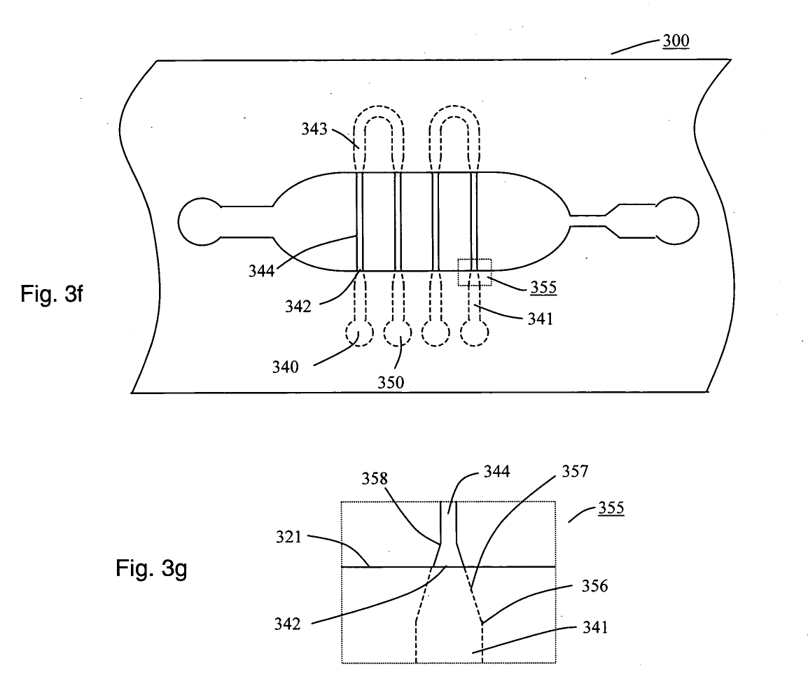

Certain embodiments of the lab-on-a-chip which use passive microfluidic control are shown in FIGS. 3a-3g. FIG. 3a shows only the detection reservoir 321 layer of the biochip 300. As shown in FIG. 3a, and FIG. 3b, the detection reservoir has a layer of considerably smaller microchannels running below it. In accordance with an embodiment of the present invention, the width 346, and depth 345 of the buried microchannels is at least 10 times less than the depth of the detection reservoir. Specifically, the width 346 and depth of the microchannels is a few μm, ranging from 1 μm to 10 μm when the detection reservoir depth is 100 μm. As shown in FIG. 3b, the buried microchannels may be fabricated using a two-step microstructure or, as shown in FIG. 3c, the narrower microchannels may be fabricated on a different substrate 306 which is bonded to the detection reservoir substrate 304.

When a liquid is introduced into the inlets 340 of these microchannels, a strong capillary force (due to the extremely small channel dimensions) sucks the liquid along the microchannel 341. At the intersection of the narrow channel with the detection reservoir, the liquid will preferentially continue to flow in the narrow channels only, due to the same capillary forces. This effect can be used to confine the flow in a given microchannel so that multiple channels, each containing a different capture antibody solution, may be made to flow through parallel channels. The primary antibody solutions may be introduced during the biochip manufacturing process (after the biochip is completely assembled). After incubation, the biochip is maintained at room temperature for an extended period of time (approximately few tens of minutes). Due to the small volumes, the liquid in the microchannels will evaporate quickly leaving behind the primary antibody coated in the narrow channels. Alternately, after incubation, the biochip may be immediately frozen to preserve the capture antibodies in solution. Thus, using this method, it is possible to easily deposit an array of capture antibodies in precise locations along the detection reservoir for simultaneous detection of an array of antigens.

As seen from the above discussion, the primary effect governing the filling characteristics is the surface tension force along the width and depth of the microchannel at the front end of the liquid meniscus as it is being sucked in to the biochip. The use of narrower (and shallower) channels will allow for a much stronger capillary force at the meniscus since the capillary force is inversely related to the microchannel dimensions.

Also, this idea may be extended to various other designs based on the concept disclosed in FIG. 3a. For example, FIG. 3d shows another embodiment of this concept wherein, the microchannel loops back and forth such that each capture antibody is presented at multiple locations 344 along the detection reservoir. This may be used for verification of the immunoassay wherein results from each of the microchannels are compared and/or averaged to get the final results. FIG. 3e shows yet another embodiment of this idea wherein, a continuous flow path with varying widths 346, 347, 348, and 349 is used for the capture antibody solution. In this case, the absolute number of capture antibodies bound at the different width sections will be different. This type of arrangement may be particularly useful for threshold detection applications, wherein the highest signal (for a particular concentration of the target antigen) will be generated in the widest section 374, and the signal will successively decrease to the lowest signal in the narrow section 344. Limits may be determined to establish the detectable target antigen concentration from each channel and these limits may be used for estimating the range of concentration of the target antigen. Yet another embodiment of this design is shown in FIGS. 3f to 3g. In this design, the microchannel 341 is further reduced in width using a taper section 357. The taper is started at a point 356 before the intersection 342 of the buried channel and the detection reservoir 321. The taper is continued a short distance within the detection reservoir 358 before terminating into a narrower section 344. The taper causes further concavity in the advancing liquid meniscus which leads to higher capillary forces. Furthermore, the detection zone may be confined to a narrower region using this design.

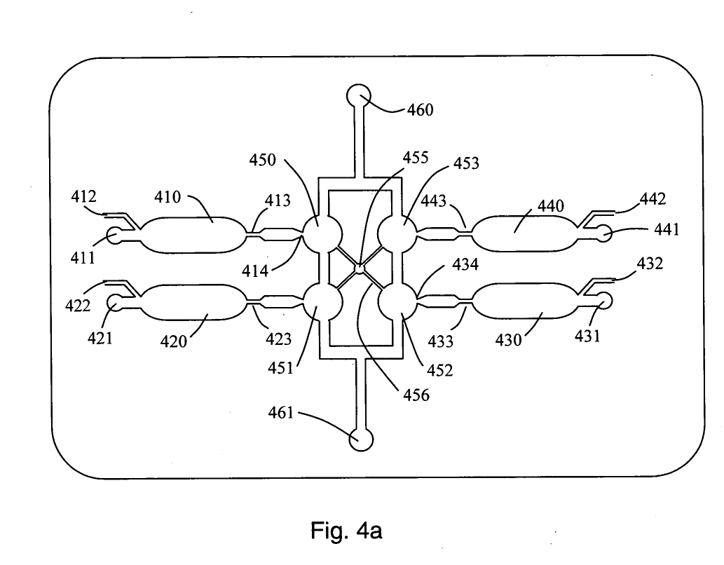

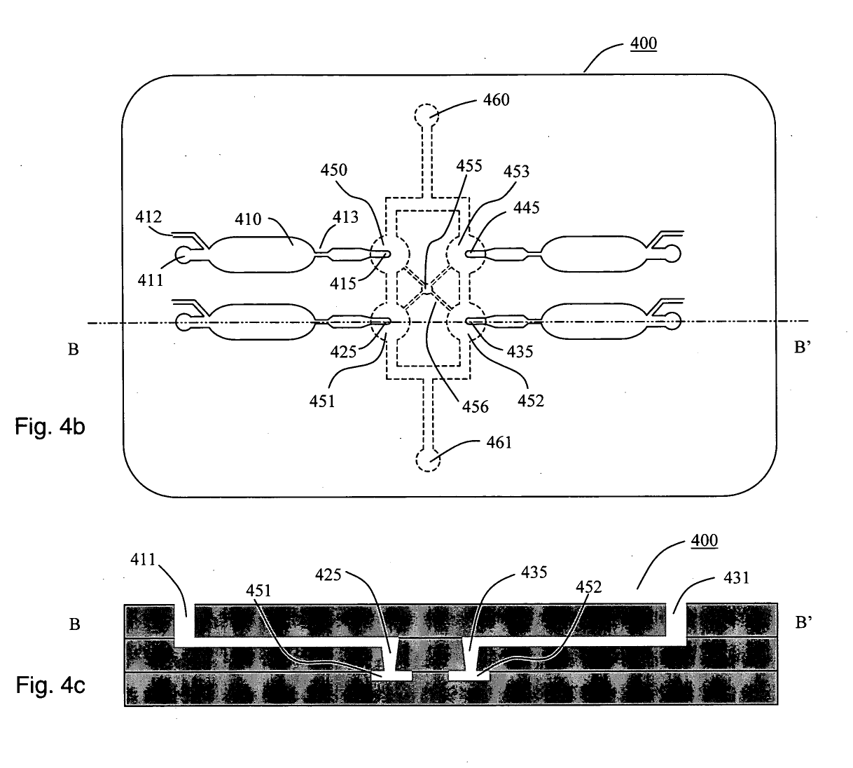

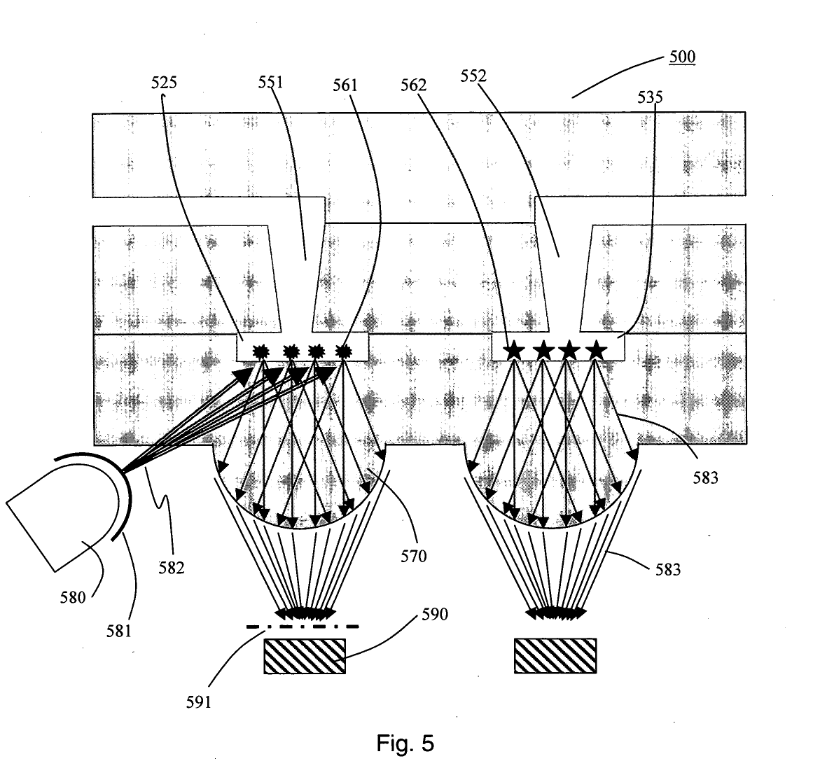

FIG. 4a shows yet another design that is well suited for lab-on-a-chip type protein analysis devices, in accordance with an embodiment of the present invention. In this embodiment, the primary antibody solutions are loaded into microdispenser reservoirs 410, 420, 430, and 440 through self sealing inlets 411, 421, 431, and 441. Passive valves 413, 423, 433, and 443 confine the liquid to the reservoir. The biochip is then sealed and frozen to preserve the antibodies and to prevent evaporation. During actual biochip operation, the first step (after sample injection) is to deliver the primary antibodies to the detection areas 450, 451, 452, and 453 by applying pressure (using solid propellant actuators) through the air inlets 412, 422, 432, and 442 at each dispensing reservoir (note that the volume of the detection chambers is matched to the volume of the dispensing reservoirs even if this is not apparent from the scales of FIGS. 4a and 4b). Constrictions 414 at the intersection of the dispensing channel and the detection area are used to ensure that there is no reverse flow in to the reservoirs when the remaining assay components are being introduced in the detection area. The microdispensing scheme used here is demonstrated clearly in U.S. patent application Ser. No. 10/602,575 filed on Jun. 24, 2003 which is incorporated herein by reference in its entirety. The air vent 455 allows the air within the detection areas to escape such that there is no pressure transfer within the detection zones that might inhibit proper filling. The width of the channels 456 leading to the air vent is very small to ensure a high flow resistance thereby ensuring that there is no liquid leakage from the air vent. Following the introduction and incubation of the capture antibodies, the remaining assay components are sequentially introduced via a common wash channel 460.

This idea may be extended even further to the 3D configuration shown in FIG. 4b, in accordance with an embodiment of the present invention. In this case, the dispensing reservoirs 410 are coupled with microfabricated nozzles 415, which are used to transfer the precisely dispensed volume to another layer which houses the wash channel. De-coupling the two microfluidic structures increases the fabrication complexity but will considerably simplify the biochip operation and make it more reliable. This is owing to the fact that the remaining immunoassay components are less likely to “backflow” into the capture antibody reservoirs because of the significant flow resistance offered by the nozzles.

FIG. 5 shows a technique to improve the detection sensitivity by the use of an integrated microlens array, in accordance with an embodiment of the present invention. Plano-Convex microlenses are fabricated on the backside of the detection chambers. FIG. 5 shows the schematic scheme for fluorescent detection and chemiluminescence detection. As shown in FIG. 5 for fluorescent detection, the fluorescent tags 561 on the secondary antibodies are exposed to excitation light 582 from a light source 580 filtered through an appropriate filter 581. Each of the fluorescent molecules will absorb the incident energy and re-emit light omni-directionally 583 at another wavelength. The light is measured by an appropriate photo detector 590 after passing through yet another filter 591. As can be seen from FIG. 5, if the lens is not used, only a small fraction of the fluorescent signal will arrive at the photodetector 590. However, the lens 570 focuses most of the fluorescent signal onto the detector thereby increasing the sensitivity of detection. The schematic for chemiluminescence is illustrated on the right hand section of FIG. 5. As shown, no filters are needed for chemiluminescence since only one wavelength (i.e. the emission wavelength) is involved in the optical system and the emission is a result of a chemical reaction thus eliminating the need for any excitation sources. The microlens array shown in FIG. 5 may be fabricated as an integrated part of the biochip using the fabrication techniques described in U.S. Patent Application having attorney document number 200057.00012, incorporated herein in its entirety by reference.

The aforementioned techniques offer numerous advantages for the development of a disposable lab-on-a-chip for point-of-care detection of protein biomarkers, a few of which are enumerated hereafter.

Certain embodiments of the present invention allow the fabrication of a low-cost protein biochip on a plastic substrate using mass-producible manufacturing techniques.

Certain embodiments of the present invention provide the ability to pattern the locations of capture antibody adsorption easily using a selective surface modification process.

Certain embodiments of the present invention provide the ability to control the location of the capture antibody adsorption precisely in the detection region using passive microfluidic controls.

Certain embodiments of the present invention provide the ability to control antibody patterning by using integrated microdispenser arrays.

Certain embodiments of the present invention provide disposable biochips having the ability to increase the sensitivity of detection by use of a microlens array.

Certain embodiments of the present invention provide the ability to manufacture a fully integrated biochip having minimal controls for microfluidic sequencing and biochip operation.

Certain embodiments of the present invention provide biochips which may be used for point-of-care testing applications.

Certain embodiments of the present invention provide the ability to simultaneously measure multiple antigens.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method to fabricate a protein micro-array used in a biochip, said method comprising selectively modifying a surface of a plastic substrate to generate a modified surface having a plurality of hydrophobic spots on a hydrophilic background.

2. The method of claim 1 wherein said plastic substrate comprises one of cyclic olefin copolymer, polymethylmethaacrylate, and polycarbonate.

3. The method of claim 1 wherein said selectively modifying comprises:
performing photolithography to distinguish said plurality of spots from said background on said surface; and
performing reactive ion etching to change a contact angle of said surface such that said plurality of spots are hydrophobic and said background is hydrophilic.

4. The method of claim 3 wherein said performing reactive ion etching includes using a reactive plasma in a reactive ion etching system.

5. The method of claim 4 wherein said reactive plasma comprises O₂ plasma.

6. The method of claim 1 further comprising exposing said modified surface of said plastic substrate to a solution of antibody molecules such that said antibody molecules selectively adsorb to said hydrophobic spots.

7. The method of claim 6 wherein said exposing comprises:
applying said solution of antibody molecules to said modified substrate;
incubating said substrate for a predetermined period of time;
rinsing said incubated substrate with a buffer solution; and
drying said rinsed substrate with an inert gas.

8. The method of claim 6 further comprising evaluating said adsorption of said antibody molecules to said exposed plastic substrate.

9. The method of claim 8 wherein said evaluating comprises:
applying a solution of fluorescently-labeled antigen for a predetermined period of time to said substrate;
rinsing said substrate with a buffer solution;
drying said rinsed substrate with an inert gas; and
observing said dried substrate with a fluorescence scanner.

10. A protein micro-array used in a biochip, said micro-array comprising a plastic substrate having a selectively modified surface wherein said selectively modified surface includes a plurality of hydrophobic spots arranged on a hydrophilic background.

11. The protein micro-array of claim 10 wherein said plastic substrate comprises one of cyclic olefin copolymer, polymethylmethaacrylate, and polycarbonate.

12. The protein micro-array of claim 10 further comprising a plurality of antibody molecules being selectively adsorbed to said hydrophobic spots.

13. The protein micro-array of claim 12 further comprising at least one antigen molecule being conjugated with each of said plurality of antibody molecules.

14. The protein micro-array of claim 13 further comprising at least one fluorescent-tagged molecule or enzyme-tagged molecule being chemically bound to said at least one antigen molecule.

15. An integrated protein biochip, said biochip comprising:
a protein micro-array; and
a micro-fluidic system to transport fluids to said protein micro-array.

16. The biochip of claim 15 wherein said protein micro-array and said micro-fluidic system are fabricated on a single plastic substrate.

17. The bio-chip of claim 15 wherein said micro-fluidic system comprises a dispensing subsystem and a washing subsystem.

18. The bio-chip of claim 17 wherein said protein micro-array and said washing subsystem are fabricated on a first plastic substrate and said dispensing subsystem is fabricated on a second plastic substrate.

19. The biochip of claim 17 wherein said dispensing subsystem comprises an array of micro-nozzles to dispense said fluids onto hydrophobic spots of said protein micro-array.

20. The biochip of claim 15 wherein said protein micro-array comprises a plastic substrate having a selectively modified surface wherein said selectively modified surface includes a plurality of hydrophobic spots arranged on a hydrophilic background.

21. The biochip of claim 15 wherein said fluids comprise at least one of an antibody solution, an antigen solution, a buffer solution, and a solution of antibody molecules tagged with at least one of fluorescent molecules and enzymes.

22. The biochip of claim 17 wherein said dispensing subsystem comprises a plurality of micro-dispensers and micro-channels connecting to said protein micro-array.

23. The biochip of claim 17 wherein said washing subsystem comprises a plurality of micro-channels connecting to said protein micro-array.

24. The biochip of claim 15 further comprising an optical micro-lens array aligned with said protein micro-array to enhance detection of at least one of fluorescent-tagged molecules and enzyme-tagged molecules associated with said protein micro-array.

25. The biochip of claim 16 wherein said plastic substrate comprises one of cyclic olefin copolymer, polymethylmethaacrylate, and polycarbonate.

26. The biochip of claim 18 wherein said first plastic substrate and said second plastic substrate comprise at least one of cyclic olefin copolymer, polymethylmethaacrylate, and polycarbonate.

27. A method to fabricate an integrated protein biochip, said method comprising:
fabricating a protein micro-array having a plurality of hydrophobic spots on a hydrophilic background; and
fabricating a micro-fluidic system connecting to said protein micro-array to deliver fluids to said plurality of hydrophobic spots of said protein micro-array.

28. The method of claim 27 further comprising fabricating an optical micro-lens array and aligning said optical micro-lens array with said protein micro-array to enhance detection of at least one of fluorescent-tagged molecules and enzyme-tagged molecules associated with said protein micro-array.

29. The method of claim 27 further comprising:
delivering a first antibody solution to at least one of said plurality of hydrophobic spots via said micro-fluidic system;
incubating said biochip for a predetermined period of time such that a plurality of antibody molecules of said first antibody solution selectively adsorb to said at least one of said plurality of hydrophobic spots; and
delivering a first buffer solution to said at least one of said plurality of hydrophobic spots via said micro-fluidic system such that any non-adsorbed portion of said first antibody solution is rinsed from said at least one of said plurality of hydrophobic spots.

30. The method of claim 29 further comprising:
delivering a target protein solution to said at least one of said plurality of hydrophobic spots via said micro-fluidic system such that a plurality of target protein molecules of said target protein solution conjugate with said plurality of adsorbed antibody molecules; and
delivering a second buffer solution to said at least one of said plurality of hydrophobic spots via said micro-fluidic system such that any non-conjugated portion of said target protein solution is rinsed from said at least one of said plurality of hydrophobic spots.

31. The method of claim 30 further comprising evaluating said biochip for adsorption of said antibody molecules and conjugation of said target protein molecules.

32. The method of claim 31 wherein said evaluating comprises:
delivering a fluorescently-labeled antibody solution to said at least one of said plurality of hydrophobic spots via said micro-fluidic system such that a plurality of fluorescently-labeled antibody molecules of said fluorescently-labeled antibody solution conjugate with said plurality of target protein molecules;
delivering a third buffer solution to said at least one of said plurality of hydrophobic spots via said micro-fluidic system such that any non-conjugated portion of said fluorescently-labeled antibody solution is rinsed from said at least one of said plurality of hydrophobic spots; and
observing said fluorescently-labeled antibody molecules of said at least one of said plurality of hydrophobic spots using a fluorescence scanner.

33. The method of claim 27 wherein said protein micro-array and said micro-fluidic system are fabricated on at least one plastic substrate.

34. The method of claim 33 wherein said at least one plastic substrate comprises at least one of cyclic olefin copolymer, polymethylmethaacrylate, and polycarbonate.
